(19) United States
(12) Patent Application Publication
(10) Pub. No.: US 2008/0038229 A1
(43) Pub. Date: Feb. 14, 2008

(54) Intracoronary injection of a mixture of autologous bone marrow derived mononuclear cells and autologous bone marrow derived mesenchymal stem cells for utilization and rescue of infarcted myocardium

(76) Inventors: Jose J. Minguell, Santiago (CL); Gabriel Perez Lasala, Covington, LA (US)

Correspondence Address: TCA CELLULAR THERAPY, LLC;ATTN: VICKI L. NICELY, 803 TRADITION DRIVE, COVINGTON, LA, 70433, US

(21) Appl. No.: 11/500,317

(22) Filed: Aug. 8, 2006

(51) Int. Cl. A61K 35/14

(52) U.S. Cl. 424/93.7

(57) ABSTRACT

The present invention is a method for improving cardiac function and myocardial regeneration in living subjects after the occurrence of myocardial infarction. The method is a combination stem cell therapy involving a mixture of bone marrow-derived mesenchymal stem cells and bone marrow derived mononuclear cells surgically implanted by using either a direct or catheter-mediated injection into damaged myocardium. Studies have shown that the implant improves heart function and myocardial regeneration as assessed by MRI, SPECT and echocardiographic measurements.

### BACKGROUND OF THE INVENTION

### Technical Field

Myocardial dysfunction resulting from atherosclerosis related myocardial infarction (MI) is a widespread and important cause of morbidity in the USA and mortality amongst adults. Due to scar- and ischemia-related post infarction events, clinical manifestations are enormous and heterogeneous. The damaged left ventricle undergoes progressive ‘remodeling’ and chamber dilation, with myocyte slippage and fibroblast proliferation. These events reflect an apparent lack of effective intrinsic mechanisms for myocardial repair and regeneration. Unless, deep (and still unknown) modifications are introduced in the area proximate to the damage to force proliferation of resident myocytes (Beltrami, 2001), all restorative therapies for MI must consider the use of an exogenous source of cardiomyocyte progenitors.

A main issue in the decision to be taken has been the source and nature of cells to utilize. According to preclinical studies, the choice has ranged from resident differentiated but quiescent cardiomyocytes to stem cells or cardiomyocyte progenitors (Warejcka, 1996; Wang, 2000; Siminiak, 2003). Since, a cardiac monopotential stem cell has not yet been identified, the clinical options are narrowed to the use of a multipotential stem cell exhibiting a potential to differentiate into the cardiomyocyte lineage. From this point of view, marrow-located stem cells display the required biological properties for a cell therapy approach to treat patients with myocardial infarction (Wulf, 2001; Wagers, 2002; Herzog, 2003). Using animal models, it has been reported a near-normalization of ventricular function after treatment of acute infarcted myocardium with locally-injected bone marrow-derived precursor cells (Jackson, 2001; Orlic, 2001, for a recent review, see Husnain, 2005). However, it was not clear whether the beneficial effect produced by the graft was elicited by hematopoietic stem cells, precursors for cardiomyocytes and/or endothelial cells, stem cell plasticity or just contamination with other marrow cells (Wagers, 2002). On the other hand, the transplantation of unfractionated sheep bone marrow into chronically infarcted myocardium did not result in any beneficial effect (Bel, 2003).

In addition, several studies have utilized mesenchymal stem cells (MSC) as a cell archetype for regenerative purposes after myocardial infarction. In vitro studies have shown that MSC have the potential to differentiate into spontaneous beating myotube-like structures, which express natriuretic peptides, myosin, desmin, and actinin and exhibit sinus node-like and ventricular cell-like action potentials (Makino, 1999; Bittira, 2002). In vivo studies have shown that when MSC are implanted into myocardium they undergo a milieu-dependent (microenvironment) cardiomyogenic differentiation and develop into myofibers containing striated sarcomeric myosin heavy chain and cell to cell junctions (Wang, 2000; Barbash, 2003). The xenogeneic or syngeneic transplantation of MSC have shown that infused cells were signaled and recruited to the normal and/or injured heart (Allers, 2004; Bittira, 2002), where they undergo differentiation and participate in the pathophysiology of post-infarct remodeling, angiogenesis and maturation of the scar (Bittira, 2003; Pittenger, 2005; Minguell, 2006). Furthermore, recent pig studies have shown that MSC infusion improves left ventricular function following myocardial infarction with no detectable immune or other toxicity (Min, 2002; Shake, 2002).

Thus, the results of experimental studies showing that the implant of bone marrow-derived progenitor cells improves heart function after myocardial infarction have prompted several groups to test this notion in people. In the last 3 years, various clinical studies have assessed the effect of transplantation of autologous bone marrow in myocardial regeneration after acute myocardial infarction. In all these studies, the source of “repairing” cells has been the bone marrow mononuclear cell fraction (BM-MNC), which contains B, T and NK lymphocytes, early myeloid cells, endothelial progenitors and a very low number of hematopoietic and/or mesenchymal stem cells. In these studies, bone marrow was aspirated (40-250 ml) from patients, the BM-MNC prepared and the resulting cells (10⁶ to 10⁷) implanted into the infarcted ischemic myocardium, by using either a direct or a catheter-mediated injection. Results showed that the autologous implantation procedure is safe, feasible and seems to be effective under clinical conditions (Assmus, 2002; Perin, 2003; Sekiya, 2002; Stamm, 2003; Strauer, 2002; Tse, 2003). In all cases, the observed therapeutic effect was attributed to bone marrow progenitors-associated neovascularization (angiogenesis, Rafii, 2003), thus improving perfusion of infarcted myocardium.

Based on preclinical and clinical studies, the rationale of the present clinical study is the following: every clinical attempt for myocardial regeneration might consider the implant of autologous progenitor cells, with the potential to differentiate and mature into cardiomyocytes, thus contributing to the recovery of local contractility. However, a comprehensive therapy should also consider the revascularization of the ischemic tissue by the implant of endothelial progenitor cells.

### BRIEF SUMMARY OF INVENTION

Consequently, we propose that the combined infusion of autologous purified and expanded marrow-derived mesenchymal stem cells (a source of cardiomyocyte progenitor) and autologous bone marrow mononuclear cells (a primary source of endothelial progenitors) represents an effective and enduring myocardial replacement therapy. The above presupposes that the pair of implanted autologous progenitors will express their respective biological programs after interacting with proper microenvironment locus of the receptor tissue (Minguell, 2001; Wagers, 2002; Rafii, 2003).

### DETAILED DESCRIPTION OF THE INVENTION

Results of experimental studies have shown that intramyocardial implantation of autologous mononuclear bone marrow cells induces neovascularisation, but not a robust improvement in heart function, after myocardial infarction. We propose that the above therapy in conjunction with one that provides a source of cardiomyocytes will represent a substantial promise as a cellular agent for cardiovascular therapy.

As a source of cardiomyocyte progenitors and based on in vitro, ex vivo and in vivo studies, we propose the use of autologous ex vivo expanded bone marrow-derived mesenchymal stem cells (MSC). Encouraging preliminary efficacy data in large animal models of myocardial infarction (Minguell, 2006) and accumulating safety data from human studies of MSCs in non-cardiovascular applications is encouraging.

In detail, our invention is the intracoronary injection (implant via catheter or direct injection) of a mixture of autologous bone marrow-derived mesenchymal stem cells (BM-MSCs) (cells that have the potential to differentiate and mature into mature cardiomyocytes) and autologous bone marrow-derived mononuclear cells (BM-MNCs) (cells that contain endothelial progenitors) that have the potential to differentiate and mature into cardiomyocytes and endothelial cells, representing an effective and enduring myocardial replacement therapy. See procedure below.

Primary bone marrow aspirations from the iliac crest will be performed in patients twenty-five±five days before receiving the cell infusion for preparation and expansion of BM-MSC. A secondary (25±5 days from primary aspiration) bone marrow aspiration from the iliac crest for preparation of BN-MNC will be performed within 5 hours of the intracoronary cell infusion to patients. For cell infusion, aliquots of autologous expanded BM-MSC and BM-MNC are taken and mixed together for a final volume of infusion medium.

For a better understanding of procedures and schedule, please refer to the following Table.

TABLE 1
DIAGRAM OF PROCEDURES AND SCHEDULE

| Days to | | Type of sample | |
|---|---|---|---|
| infusion | Step | to be taken | Type of test to be performed |
| −25 | 1^{st} Bone marrow aspirate for | cell suspension | differential cell count; |
| | preparation of MSC cells | | microbiological |
| −25 | Mononuclear cell fraction | cell suspension | differential cell count |
| −20 | Passage # 0 (Primary BM-MSC | growth medium | cell number, viability, |
| | culture) | & cell | microbiological |
| | | suspension | |
| −16 | Passage #1 | cell suspension | cell number, viability |
| −12 | Passage #2 | cell suspension | cell number, viability |
| −8 | Passage #3 | cell suspension | cell number, viability |
| −4 | Passage #4 (Expanded MSC) | growth medium | cell number, viability, |
| | | & cell | microbiological, mycoplasma, |
| | | suspension | |
| 0 | Final preparation of | BM-MSC | cell number, viability |
| | BM-MSC | suspension | microbiological, mycoplasma, |
| | | | Gram stain, immunotypification, |
| | | | differentiation potential |
| 0 | 2^{nd} Bone marrow aspirate for | BM-MNC | cell number, viability, |
| | preparation of MNC cells | Suspension | microbiological, Gram stain |
| | | | immunotypification, |
| 0 | Cell product for infusion (final | BM-MSC plus | cell number, viability, |
| | mixture of autologous BM- | BM-MNC | microbiological, Gram stain, |
| | MSC and BM-MNC) | suspension | endotoxin, |

BM-MNC: bone marrow-derived mononuclear cell fraction
BM-MSC: bone marrow-derived mesenchymal stem cells

Cell infusion (transplantation) may be done in patients intraoperatively in conjunction with coronary artery bypass grafting by direct injection following the circumference of the infarct border or via intracoronary percutaneous balloon catheter designed for angioplasty. Subjects may include patients who fit criteria for acute myocardial infarction or patients with a defined region of myocardial dysfunction related to a previous myocardial infarction.

Wall motion and left ventricular ejection fraction is evaluated by MRI and echocardiography. SPECT is used to assess viability and myocardial perfusion.

### REFERENCES

Allers C, Sierralta W D, Neubauer S, Rivera F, Minguell J J, Conget P A. Dynamic of distribution of human bone marrow-derived mesenchymal stem cells after transplantation into adult unconditioned mice. Transplantation 78, 503, 2004

Assmus B, Schachinger V, Teupe C, Britten M, Lehmann R, Dobert N, Grunwald F, Aicher A, Urbich C, Martin H, Hoelzer D, Dimmeler S, Zeiher A M. Transplantation of Progenitor Cells and Regeneration Enhancement in Acute Myocardial Infarction (TOPCARE-AMI). Circulation 2002; 06: 3009-3017.
Barbash I M, Chouraqui P, Baron J et al. Systemic delivery of bone marrow-derived mesenchymal stem cells to the infarcted myocardium. Circulation. 2003; 108: 863.
Beltrami A P, Urbanek K, Kajstura J, Yan S M, Finato N, Bussani R, Nadal-Ginard B, Silvestri F, Leri A, Beltrami C A, Anversa P. Evidence that human cardiac myocytes divide after myocardial infarction. N Engl J Med. 2001; 344:1750-1757.
Bittira B, Kuang J Q, Al-Khaldi A, Shum-Tim D, Chiu R C. In vitro pre-programming of marrow stromal cells for myocardial regeneration. Ann Thorac Surg. 2002; 74: 1154-1159.
Bittira B, Shum-Tim D, Al-Khaldi A, Chiu R C. Mobilization and homing of bone marrow stromal cells in myocardial infarction. Eur J Cardiothorac Surg. 2003; 24: 393-398.
Herzog E L, Chai L, Krause D S. Plasticity of marrow-derived stem cells. Blood 2003; 102: 3483-3493.
Husnain H K, Ashraf M. Bone marrow stem cell transplantation for cardiac repair. Am J Physiol Heart Circ Physiol 2005; 288: H2557-H2567.
Jackson K A, Majka S M, Wang H, Pocius J, Hartley C J, Majesky M W, Entman M L, Michael L H, Hirshi K K, Godell M A. Regeneration of ischemic cardiac muscle and vascular endothelium by adult stem cells. J Clin Invest 2001; 107: 1395-1402
Makino S, Fukuda K, Miyoshi S, Konishi F, et al. Cardiomyocytes can be generated from marrow stromal cells in vitro. J Clin Invest. 1999; 103: 697-705.
Minguell J J, Erices A, Conget P. Mesenchymal stem cells. Exp. Biol. Med. 2001; 226, 507-517.
Minguell J J, Erices, A. Mesenchymal Stem Cells and the Treatment of Cardiac Disease. Experimental Biology and Medicine (in press) January issue, 2006.
Min J Y, Sullivan M F, Yang Y, Zhang J P, Converso K L, Morgan J P, Xiao Y F. Significant improvement of heart function by cotransplantation of human mesenchymal stem cells and fetal cardiomyocytes in postinfarcted pigs. Ann Thorac Surg. 2002, 74: 1568-1575.
Orlic D et al. Bone marrow cells regenerate infarted myocardium. Nature 2001; 410, 701-705.
Perin E C, Dohmann H F, Borojevic R, Silva S A, Sousa A L, et al. Transendocardial, autologous bone marrow cell transplantation for severe, chronic ischemic heart failure. Circulation. 2003; 107:2294-2302
Pittenger M F, Martin B J. Mesenchymal stem cells and their potential as cardiac therapeutics. Circ Res. 2004; 95:9-20.
Rafii S, Lyden D. Therapeutic stem and progenitor cell transplantation for organ vascularization and regeneration. Nat. Med. 2003; 9: 702-712.
Sekiya, 2002 I, Larson B L, Smith J R, Pochampally R, Cui J G, Prockop D J. Expansion of human adult stem cells from bone marrow stroma: conditions that maximize the yields of early progenitors and evaluate their quality. Stem Cells, 2002; 20: 530-541.
Shake J G, Gruber P J, Baumgartner W A, Senechal G, Meyers J, Redmond J M, Pittenger M F, Martin B J. Mesenchymal stem cell implantation in a swine myocardial infarct model: engraftment and functional effects. Ann Thorac Surg. 2002; 73: 1919-1925.
Siminiak T, Kurpisz M. Myocardial replacement therapy. Circulation 2003; 108:1167-1171
Stamm C, Westphal B, Kleine H D et al. Autologous bone-marrowtem-cell transplantation for myocardial regeneration. Lancet, 2003; 361: 45-46
Strauer B E, Brehm M, Zeus T et al. Repair of infarcted myocardium by autologous intracoronary mononuclear bone marrow cell transplantation in jumans. Circulation 2002; 106: 1913-1918
Tse H F, Kwong Y L, Chan J K, Lo G, Ho C L, Lau C P. Angiogenesis in ischaemic myocardium by intramyocardial autologous bone marrow mononuclear cell implantation. Lancet. 2003; 361: 47-49.
Wagers A J, Christensen J L, Weissman I L. Cell fate determination from stem cells. Gene Therapy 2002; 9:606-612.
Wang J S, Shum-Tim D, Galipeau J, Chedrawy E, Eliopoulos N, Chiu R C. Marrow stromal cells for cellular cardiomyoplasty: feasibility and potential clinical advantages. J Thorac Cardiovasc Surg. 2000; 20: 999-1005.
Warejcka D J, Harvey R, Taylor B J, Young H E, Lucas P A. A population of cells isolated from rat heart capable of differentiating into several mesodermal phenotypes. J Surg Res 1996; 62:233-242.
Wulf G G, Jackson K A, Goodell M A. Somatic stem cell plasticity: current evidence and emerging concepts. Exp. Hematol. 2001; 29: 1361-1370

1. A safe, effective and enduring myocardial replacement therapy using a combination of autologous bone marrow-derived mesenchymal stem cells (BM-MSCs) and autologous bone marrow-derived mononuclear cells (BM-MNCs).
